(19) United States
(12) Patent Application Publication
(10) Pub. No.: US 2026/0165626 A1
(43) Pub. Date: Jun. 18, 2026

(54) EXHALATION SENSING APPARATUS, EXHALATION SENSING METHOD AND NON-TRANSITORY COMPUTER-READABLE MEDIUM

(71) Applicant: Asahi Kasei Microdevices Corporation, Tokyo (JP)

(72) Inventors: Kengo SASAYAMA, Tokyo (JP); Takaaki FURUYA, Tokyo (JP)

(21) Appl. No.: 19/412,705

(22) Filed: Dec. 8, 2025

(30) Foreign Application Priority Data
Dec. 13, 2024 (JP) 2024-219357
Oct. 6, 2025 (JP) 2025-168355

(51) Int. Cl. A61B 5/18; A61B 5/00; A61B 5/08; B60Q 9/00

(52) U.S. Cl. CPC A61B 5/18; A61B 5/082; A61B 5/4845; A61B 5/6893; A61B 5/7203; B60Q 9/00

(57) ABSTRACT

Provided is an exhalation sensing apparatus that is provided in an operator's cabin of a moving object, the exhalation sensing apparatus including: an exhalation sensing unit that senses an exhalation in a state in which an operator of the moving object is not in contact therewith; and a notification unit that notifies the operator of an instruction, wherein the exhalation sensing unit senses the exhalation before the notification unit notifies of the instruction, and the notification unit notifies the operator of the instruction based on a sensing result of the exhalation obtained by the exhalation sensing unit. The exhalation sensing unit may contain a sensing housing fixed to the moving object. The sensing housing may have an inlet through which the exhalation passes.

The contents of the following patent application(s) are incorporated herein by reference:
NO. 2024-219357 filed in JP on Dec. 13, 2024 and
NO. 2025-168355 filed in JP on Oct. 6, 2025.

### BACKGROUND

### 1. Technical Field

The present invention relates to an exhalation sensing apparatus, an exhalation sensing method, and a non-transitory computer-readable medium.

### 2. Related Art

In Patent document 1, “In a vehicle driving assistance apparatus and system, driving assistance for a driver depending on the state of the driver, such as a tendency to decrease in awareness of the driver, is performed” is described.

### RELATED ART DOCUMENTS

### Patent Document

Patent Document 1: Japanese Patent Application Publication No. 2001-219760

### BRIEF DESCRIPTION OF THE DRAWINGS

FIG. 1 is a schematic view illustrating an example of a moving object 400 mounted with an exhalation sensing apparatus 300 according to one embodiment of the present invention.

FIG. 2 is a block diagram illustrating an example of an exhalation sensing apparatus 300 according to one embodiment of the present invention.

FIG. 3 illustrates an example of a sensing housing 106.

FIG. 4 is a block diagram illustrating a configuration example of the exhalation sensing unit 10.

FIG. 5 illustrates an example of a configuration included in the moving object 400.

FIG. 6 illustrates an example of a relationship between a measurement value of a blood alcohol concentration and an alcohol concentration measured by the exhalation sensing unit 10.

FIG. 7 is a block diagram illustrating another example of the exhalation sensing apparatus 300 according to one embodiment of the present invention.

FIG. 8 illustrates an example of a correspondence between an interval between exhalations and control information.

FIG. 9 illustrates an example of a correspondence between the speed of change in exhalation volume and control information.

FIG. 10 illustrates an example of a correspondence between the interval between exhalations, the speed of change in exhalation volume, and control information.

FIG. 11 illustrates an example of a correspondence between the interval between exhalations, the speed of change in exhalation volume, and control information.

FIG. 12 is a flowchart illustrating an example of an exhalation sensing method according to one embodiment of the present invention.

FIG. 13 illustrates an example of a computer 1200 in which a plurality of aspects of the present invention may be entirely or partially embodied.

### DESCRIPTION OF EXEMPLARY EMBODIMENTS

Hereinafter, the present invention will be described through embodiments of the invention, but the following embodiments do not limit the invention according to the claims. In addition, not all of the combinations of features described in the embodiments are essential to the solving means of the invention.

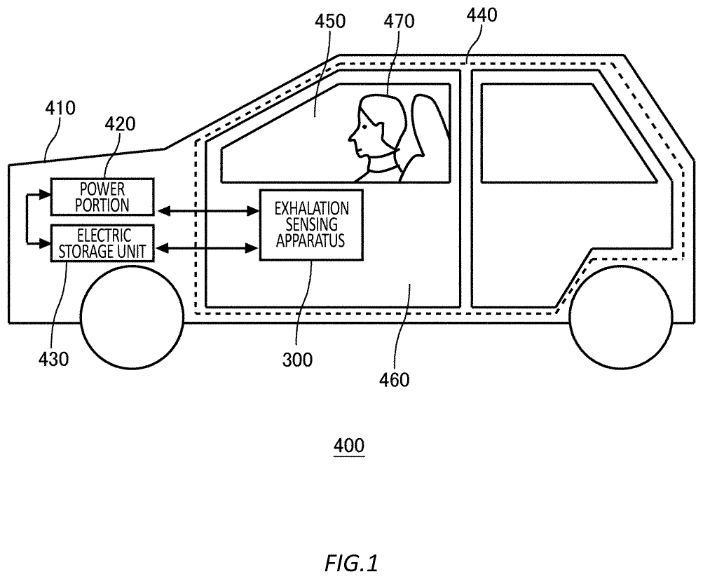

FIG. 1 is a schematic view illustrating an example of a moving object 400 mounted with an exhalation sensing apparatus 300 according to one embodiment of the present invention. The moving object 400 is an automobile, for example, but not limited thereto. The moving object 400 may be a ground moving object such as a vehicle moving on the ground, an aerial moving object such as a flying object flying in the air, a waterborne moving object such as a ship moving on the water, an underwater moving object such as a submarine boat moving underwater, or may be a moving object moving in another place.

The moving object 400 includes an operator's cabin 440 on which an operator 470 who steers the moving object 400 is getting in. The operator's cabin 440 may have a space where a passenger other than the operator 470 is getting in. The operator's cabin 440 is a space in which equipment for steering the moving object 400, such as a handle in an automobile, for example, is installed. The operator's cabin 440 of the present example is a space that is surrounded by a mobile body housing 410 of the moving object 400. The mobile body housing 410 includes a body portion of the automobile, for example. The mobile body housing 410 may include at least one of one or more windows 450, and one or more doors 460. The window 450 or the door 460 may be openable and closable between the operator's cabin 440 and an external space. Other than the window 450 and the door 460, the mobile body housing 410 may have a portion that is openable and closable.

The exhalation sensing apparatus 300 is provided in the operator's cabin 440. The exhalation sensing apparatus 300 senses an exhalation in the operator's cabin 440. The exhalation sensing apparatus 300 may sense exhalation of the operator 470, or may sense exhalation of the passenger. The exhalation sensing apparatus 300 may be an apparatus that can sense the exhalation without requiring an equipment operation by a passenger of the operator 470 or the like, or an operation such as intentional breathing by the passenger. The exhalation sensing apparatus 300 is provided partially or entirely in the operator's cabin 440.

The exhalation sensing apparatus 300 may sense an alcohol concentration (ppm). The exhalation sensing apparatus 300 may determine whether or not the sensed alcohol concentration is within tolerance. The exhalation sensing apparatus 300 may prohibit steering of the moving object 400 by the operator 470 when the sensed alcohol concentration exceeds the tolerance. When the sensed alcohol concentration exceeds the tolerance, the exhalation sensing apparatus 300 may notify the operator 470 of a warning including this fact or may notify the operator that the alcohol concentration will be re-measured.

The moving object 400 of the present example includes a power portion 420 and an electric storage unit 430. The power portion 420 generates power for moving the moving object 400. The power portion 420 may be an internal combustion engine, such as an engine that generates power by combusting fuel, for example. The power portion 420 may be an electric motor, such as a motor that rotates in response to electrical power, for example. The exhalation sensing apparatus 300 may stop generating power by the power portion 420 when the sensed alcohol concentration exceeds the tolerance.

The electric storage unit 430 accumulates electrical power. The electric storage unit 430 may supply the stored electrical power to the equipment of the moving object 400. The electric storage unit 430 may supply the electrical power to the power portion 420, may supply the electrical power to the exhalation sensing apparatus 300, or may supply the electrical power to another equipment, such as an air conditioner that adjusts the temperature of the operator's cabin 440 or a notification unit 20 that notifies the operator 470 of an instruction. The exhalation sensing apparatus 300 may stop supplying the electrical power from the electric storage unit 430 when the sensed alcohol concentration exceeds the tolerance.

The exhalation sensing apparatus 300 senses the exhalation in a state in which the operator 470 is not in contact with the exhalation sensing unit 10 (described below). The state in which the operator 470 is not in contact refers to a state in which the operator 470 is not in contact with the exhalation sensing unit 10 (described below) or a sensing housing 106 (described below) containing the exhalation sensing unit 10 (described below). The exhalation sensing apparatus 300 is a passive type. The passive type exhalation sensing apparatus 300 can sense the exhalation without intentionally blowing the exhalation by the operator 470.

An active type exhalation sensing apparatus senses the exhalation in a state in which the operator 470 is in contact with the exhalation sensing unit 10 (described below), or the sensing housing 106 (described below) containing the exhalation sensing unit 10 (described below). The active type exhalation sensing apparatus has a tube for blowing breath into the exhalation sensing apparatus. The operator 470 blows an exhalation into the tube in a state in which the operator 470 is in contact with the sensing housing 106 (described below) or the tube. The active type exhalation sensing apparatus can sense the exhalation without intentionally blowing the exhalation by the operator 470.

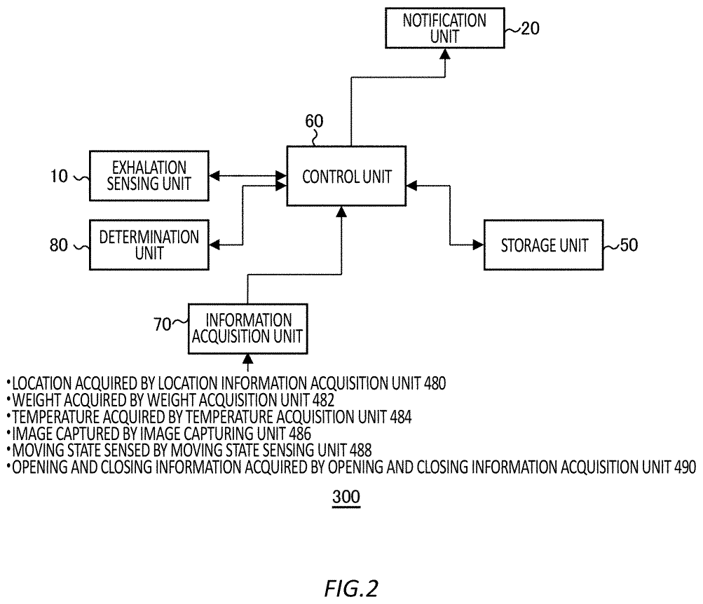

FIG. 2 is a block diagram illustrating an example of an exhalation sensing apparatus 300 according to one embodiment of the present invention. The exhalation sensing apparatus 300 includes an exhalation sensing unit 10 and a notification unit 20. The exhalation sensing apparatus 300 may include a storage unit 50, a control unit 60, an information acquisition unit 70, and a determination unit 80.

The notification unit 20 is a display, monitor, and the like, for example. The notification unit 20 may be provided on an instrument panel (instrument panel) of the moving object 400. The notification unit 20 may be a screen of a navigation of the moving object 400. The notification unit 20 may be a head-up display that is irradiated to the windshield of the moving object 400.

The exhalation sensing apparatus 300 may be partially or entirely achieved by a computer. The control unit 60 may be a Central Processing Unit (CPU) of the computer. When the exhalation sensing apparatus 300 is achieved by a computer, on the computer, an information processing program for making the computer function as the exhalation sensing apparatus 300 may be installed, or an information processing program for performing an information processing method described below may be installed.

The exhalation sensing unit 10 senses the exhalation in a state in which the operator 470 of the moving object 400 is not in contact therewith. The exhalation sensing unit 10 may sense the exhalation of the operator 470. The notification unit 20 notifies the operator 470 of an instruction. The notification unit 20 may notify the operator 470 of information for assisting measurement of the exhalation. For example, when the exhalation sensing apparatus 300 generates an exhalation sensing error, the notification unit 20 notifies the operator 470 of an instruction, such as a manner of exhalation for correctly measuring the exhalation.

The exhalation sensing unit 10 senses the exhalation before the notification unit 20 notifies the operator 470 of the instruction. As described above, the exhalation sensing apparatus 300 is a passive type. Thus, the exhalation sensing unit 10 senses the exhalation that is not intentionally blown thereto by the operator 470 before the notification unit 20 notifies the operator 470 of the instruction. However, since the operator 470 does not blow the exhalation intentionally, the exhalation sensing apparatus 300 may generate the exhalation sensing error, and the like.

The notification unit 20 notifies the operator 470 of the instruction based on the sensing result of the exhalation obtained by the exhalation sensing unit 10. For example, when the sensing result obtained by the exhalation sensing unit 10 has an error or the like, the notification unit 20 notifies the operator 470 of an instruction, such as a manner of exhalation for correctly measuring the exhalation. Thereby, the operator 470 can blow the exhalation by a guided method.

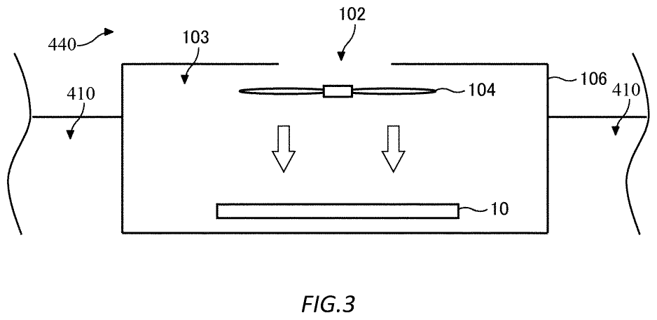

FIG. 3 illustrates an example of a sensing housing 106. The sensing housing 106 of the present example is a box-shaped housing. The sensing housing 106 is fixed on the moving object 400. The sensing housing 106 is fixed on the mobile body housing 410. The sensing housing 106 may be fixed on the operator's cabin 440 of the moving object 400. The sensing housing 106 may be fixed on the dashboard of the moving object 400, or may be fixed on the handle of the moving object 400. The exhalation sensing unit 10 may contain the sensing housing 106. In the present example, the exhalation sensing unit 10 is contained in an interior space 103 of the sensing housing 106.

The sensing housing 106 has an inlet 102. The exhalation passes through the inlet 102. The exhalation sensing unit 10 may sense the exhalation that passes through the inlet 102. The sensing housing 106 may have an air blowing unit 104. In the present example, the air blowing unit 104 is contained in the interior space 103. The air blowing unit 104 may be arranged to face the inlet 102. The air blowing unit 104 may be provided inside the sensing housing 106, or may be provided outside. The air blowing unit 104 of the present example is provided between the inlet 102 and the exhalation sensing unit 10. The air blowing unit 104 delivers the air in the operator's cabin 440 to the exhalation sensing unit 10. By driving the air blowing unit 104, the air in the operator's cabin 440 is taken from the inlet 102, and blown toward the exhalation sensing unit 10. Thereby, the exhalation sensing unit 10 easily senses the exhalation that is not intentionally blown by the operator 470.

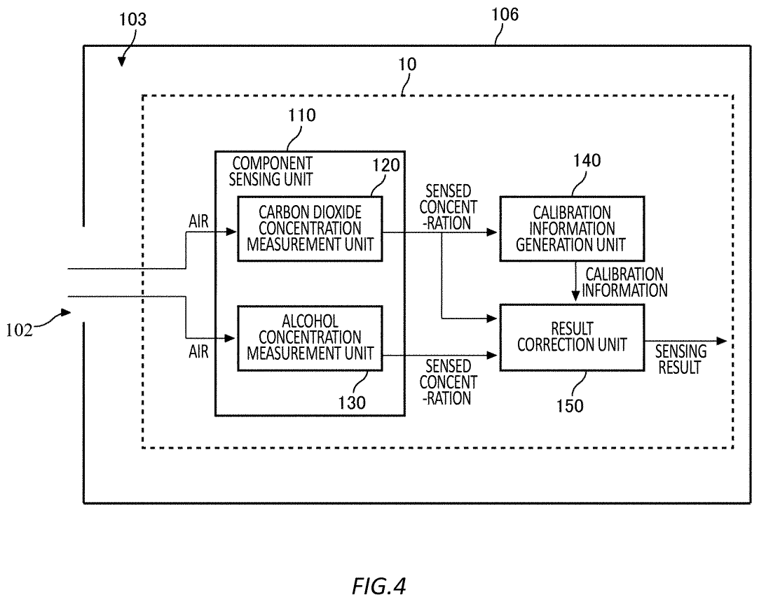

FIG. 4 is a block diagram illustrating a configuration example of the exhalation sensing unit 10. In FIG. 4, the air blowing unit 104 shown in FIG. 3 is omitted. The exhalation sensing unit 10 may include a component sensing unit 110, a calibration information generation unit 140, and a result correction unit 150.

The component sensing unit 110 senses the alcohol concentration and the concentration of carbon dioxide included in the exhalation. The component sensing unit 110 may sense sensing information indicating the alcohol concentration and sensing information indicating the concentration of carbon dioxide. Each piece of sensing information is a signal whose value changes in response to the magnitude of the concentration of each target component (in the present example, alcohol and carbon dioxide) included in the exhalation. For example, the sensing information is a signal of a value depending on the intensity of light passing through a gas included in the exhalation at a wavelength corresponding to each target component. The light intensity attenuates in response to the concentration of each target component included in the exhalation. The sensing information may be a signal obtained by converting a signal of the light into an electrical signal, or may be a signal obtained by performing a predetermined signal processing on the electrical signal. The sensing information may include the concentration value itself of each target component.

The component sensing unit 110 of the present example has a carbon dioxide concentration measurement unit 120, and an alcohol concentration measurement unit 130. The air in the operator's cabin 440 is introduced into the component sensing unit 110 via the inlet 102. The carbon dioxide concentration measurement unit 120 outputs sensing information in response to the concentration (ppm) of carbon dioxide included in the air. The carbon dioxide concentration measurement unit 120 is a non-dispersive infrared absorption (NDIR type) sensor, for example.

The alcohol concentration measurement unit 130 outputs sensing information in response to the alcohol concentration (ppm) included in the air in the operator's cabin 440. The alcohol concentration measurement unit 130 is an electrochemical (fuel cell type) sensor, for example. In the electrochemical sensor, a current generated by alcohol included in the air is sensed, for example.

The calibration information generation unit 140 generates, based on the sensing information of carbon dioxide that is sensed multiple times by the component sensing unit 110, calibration information for calibrating the concentration of carbon dioxide. The calibration information is information that is obtained by converting the value of each piece of sensing information into the concentration of each target component. The calibration information may be a calibration curve indicating a relationship between the value of the sensing information and the concentration of the target component. When the sensing information includes the concentration value itself of each target component, the calibration information may be information that corrects the concentration value in the sensing information. In the present specification, the concentration calculated from the value of the sensing information using the calibration information may be referred to as a calibrated concentration. For example, the calibration information may include a gain value that calculates the calibrated concentration by multiplying the value of the sensing information, may include a function for calculating the calibrated concentration by using the value of the sensing information as a variable, or may include a table that is obtained by associating the value of the sensing information with the calibrated concentration.

In the calibration information generation unit 140, calibration information to be used as a reference may be preset. The calibration information may be set by a manufacturer, a user, or the like of the exhalation sensing apparatus 300. The calibration information generation unit 140 may update the calibration information based on the sensing information of carbon dioxide that is sensed multiple times by the component sensing unit 110. In the present specification, the update of the calibration information may be referred to as a generation of the calibration information.

The calibration information generation unit 140 may extract sensing information whose corresponding concentration is the minimum among the sensing information of carbon dioxide sensed multiple times. In the present specification, a relative magnitude relationship between corresponding concentrations may be described as a relative magnitude relationship between pieces of sensing information. For example, among a plurality of pieces of sensing information, sensing information whose corresponding concentration is the minimum may be referred to as the minimum sensing information. The calibration information generation unit 140 may adjust the calibration information described above such that the minimum sensing information is converted into a preset reference concentration. The adjustment of the calibration information may be the adjustment of the gain value described above, may be the adjustment of each coefficient of the function, or may be the update of the table. For example, the calibration information generation unit 140 may calculate the gain value by dividing the reference concentration by the concentration corresponding to the minimum sensing information. The reference concentration corresponds to an average carbon dioxide concentration in the outside air, for example. The reference concentration may be 400 ppm or may be another value.

The carbon dioxide concentration in the operator's cabin 440 may vary depending on the exhalation of the operator 470 or the passenger. On the other hand, the carbon dioxide concentration in the operator's cabin 440 does not decrease to a level lower than the carbon dioxide concentration outside the operator's cabin 440. Thus, it can be estimated that as the value of the sensing information is lower, the closer the state in which the sensing information was measured is to the carbon dioxide concentration in the outside air. Thus, calibration information with a relatively high precision can be generated by adjusting the configuration information such that the minimum sensing information among the plurality of pieces of sensing information is converted into the reference concentration.

The result correction unit 150 corrects the sensing result of alcohol based on the calibrated concentration of carbon dioxide which is calibrated by the calibration information. The sensing result of alcohol is the alcohol concentration, for example. The result correction unit 150 corrects the alcohol concentration of a measurement target with the calibrated concentration of carbon dioxide which is measured in parallel with the alcohol concentration.

For example, the result correction unit 150 calculates, based on the calibrated concentration of carbon dioxide, a degree of dilution of the air that reached to the component sensing unit 110. The degree of dilution is an indicator indicating how much the exhalation of the operator 470 is diluted until the exhalation reaches to the component sensing unit 110 from the operator 470. The degree of dilution may be a value obtained by dividing a preset standard concentration of carbon dioxide by the calibrated concentration of carbon dioxide. The standard concentration of carbon dioxide may use the concentration of carbon dioxide included in the exhalation of an adult as an average value, or may be a value obtained by actually measuring the exhalation of the operator 470. The standard concentration of carbon dioxide is a value within a range from 1% to 9%, for example. The standard concentration of carbon dioxide may be 3%, for example. The standard concentration of carbon dioxide may be set by the manufacturer or the user of the exhalation sensing unit 10.

The result correction unit 150 may calculate a corrected alcohol concentration by multiplying the sensed alcohol concentration by the degree of dilution described above. For example, when the degree of dilution is calculated to be 150 times based on the calibrated concentration of carbon dioxide, the result correction unit 150 calculates the corrected alcohol concentration by multiplying the sensed alcohol concentration by 150 times. Thereby, the alcohol concentration included in the exhalation of the operator 470 can be estimated. In another example, the result correction unit 150 may correct, based on the degree of dilution, a threshold concentration to be compared with the sensed alcohol concentration. For example, when the degree of dilution is 150 times, the result correction unit 150 may correct the sensing result of alcohol by making the threshold concentration to be 1/150.

The exhalation sensing unit 10 of the present example calculates, from the carbon dioxide concentration, the degree of dilution which is in relation to the exhalation of the operator 470, of the air measured by the component sensing unit 110, to correct the sensing result of alcohol. Thus, the exhalation of the operator 470 may not be directly blown to the component sensing unit 110. The exhalation sensing unit 10 of the present example can measure the alcohol concentration of the operator 470 even in a state where the operator 470 is not intended to measure the alcohol concentration. Then, since calibration information of the carbon dioxide concentration is generated based on the carbon dioxide concentration (sensing information in the present example) measured multiple times inside the operator's cabin 440, a change in the characteristic of the component sensing unit 110 over time can be corrected, the degree of dilution can be calculated precisely, and the alcohol concentration can be measured precisely.

The exhalation may be introduced into the sensing housing 106 through the inlet 102 for a certain time period. The certain time period may be predetermined. The certain period of time may be 0.5 seconds or more and 2 seconds or less, or may be one second or more or 1.5 seconds or less. The exhalation may be continuously introduced into the sensing housing 106 for a certain time period.

When the exhalation is introduced into the sensing housing 106 for a certain time period, the exhalation sensing unit 10 may sense the exhalation based on the carbon dioxide concentration in the interior space 103 of the sensing housing 106. When the exhalation is introduced into the sensing housing 106 for a certain time period, the carbon dioxide concentration of the interior space 103 reflects the total amount of carbon dioxide introduced into the sensing housing 106 for a certain time period. Thus, the exhalation sensing unit 10 easily senses a gas introduced into the interior space 103 through the inlet 102 as an exhalation.

The exhalation sensing unit 10 may sense a gas introduced into the interior space 103 for a certain period of time through the inlet 102 as an exhalation when the carbon dioxide concentration of the interior space 103 is greater than a first threshold. The notification unit 20 may notify a message such as “the exhalation was sensed”. The exhalation sensing unit 10 may not sense a gas introduced into the interior space 103 through the inlet 102 as an exhalation when the carbon dioxide concentration of the interior space 103 is equal to or less than a predetermined first threshold. The notification unit 20 may notify a message such as “The exhalation cannot be sensed. Please blow your breath.”, “Please put your face closer.”, or “Please remove your mask if you are wearing a mask”.

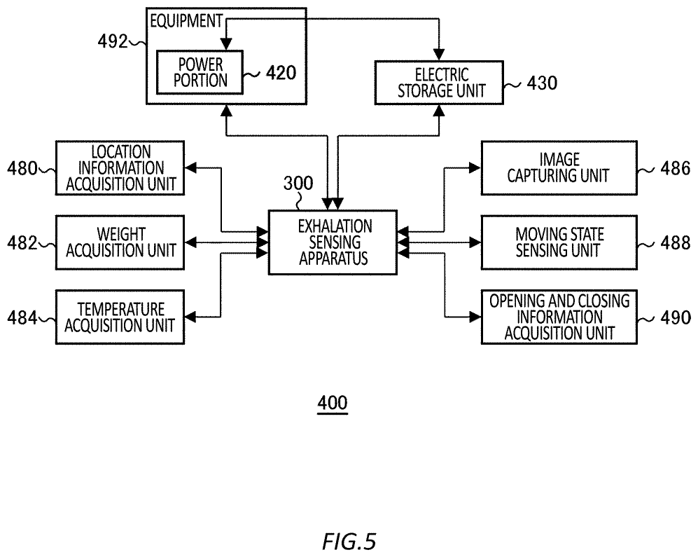

FIG. 5 illustrates an example of a configuration included in the moving object 400. The moving object 400 may have a power portion 420, an electric storage unit 430, a location information acquisition unit 480, a weight acquisition unit 482, a temperature acquisition unit 484, an image capturing unit 486, a moving state sensing unit 488, an opening and closing information acquisition unit 490, and an equipment 492. The location information acquisition unit 480, the image capturing unit 486, the moving state sensing unit 488, the opening and closing information acquisition unit 490, and the equipment 492 may be provided in the mobile body housing 410. The weight acquisition unit 482 may be provided on a seat portion of a seat on which the operator 470 is to be seated. The temperature acquisition unit 484 and the image capturing unit 486 may be provided in the mobile body housing 410 on the front of the operator 470. The temperature acquisition unit 484 may be provided on a part of a handle, with which the hand of the operator 470 is to be in contact.

The exhalation sensing unit 10 (see FIG. 2) may sense alcohol included in the exhalation. The notification unit 20 may notify the operator 470 of an instruction based on the sensing result of alcohol in the exhalation. The sensing result of alcohol may be a sensing result of alcohol that is corrected by the result correction unit 150.

The location information acquisition unit 480 acquires the location information of the moving object 400. The location information acquisition unit 480 is, for example, a global positioning system (GPS). The information acquisition unit 70 of the exhalation sensing apparatus 300 acquires the location acquired by the location information acquisition unit 480 of the moving object 400. The information acquisition unit 70 may acquire the location wirelessly.

The exhalation sensing unit 10 may determine the alcohol concentration in the exhalation, and a magnitude relationship between reference values of alcohol at the location acquired by the location information acquisition unit 480. A reference of the alcohol concentration determined as drunk driving may be different for each country or region. In the storage unit 50 (see FIG. 2), the reference of the alcohol concentration determined as the drunk driving may be pre-stored for each country or region. The exhalation sensing unit 10 may acquire, based on the reference of the alcohol concentration for each country or region stored in the storage unit 50, a reference value of alcohol concentration at a location acquired by the location information acquisition unit 480. The exhalation sensing unit 10 may determine which is greater between the reference value of the acquired alcohol concentration and the sensed alcohol concentration.

The notification unit 20 may notify the operator 470 of an instruction, based on the alcohol concentration in the exhalation, and a magnitude relationship between reference values of the alcohol concentration at the location acquired by the location information acquisition unit 480. For example, the notification unit 20 notifies the operator 470 of a message such as “There is a possibility of drunk driving. Please immediately stop the car.” when the alcohol concentration in the exhalation is equal to or greater than the reference value. When the alcohol concentration of the exhalation is less than the reference value, the notification unit 20 may notify the operator 470 of a message such as “not drunk driving.” or may not notify the message.

The temperature acquisition unit 484 acquires the temperature of the operator 470. When the operator 470 is a human, the temperature acquisition unit 484 acquires the body temperature of the human. The temperature acquisition unit 484 is a thermographic camera, for example. The temperature acquisition unit 484 may be a touch-sensitive thermometer provided on the handle of the moving object 400.

The information acquisition unit 70 of the exhalation sensing apparatus 300 acquires the temperature acquired by the temperature acquisition unit 484 of the moving object 400. The information acquisition unit 70 may acquire the temperature wirelessly. The body temperature of a human easily increases when the human takes alcohol. A threshold of the body temperature of a human who is determined to be drunk may be predetermined. The body temperature is 37.0° C., for example. When the body temperature is greater than a body temperature threshold, it may be determined that the human is drunk.

The notification unit 20 notifies the operator 470 of an instruction based on the sensing result of alcohol, and the temperature acquired by the temperature acquisition unit 484. For example, the notification unit 20 notifies the operator 470 of a severe warning when the exhalation sensing unit 10 senses alcohol and the temperature acquired by the temperature acquisition unit 484 is equal to or greater than the body temperature threshold. The severe warning is a notification such as “Please stop the car immediately now”, for example. The notification unit 20 may issue a warning sound together with a notification by means of text.

For example, the notification unit 20 notifies the operator 470 of a minor warning when the exhalation sensing unit 10 does not sense alcohol and the temperature acquired by the temperature acquisition unit 484 is equal to or greater than the body temperature threshold. The body temperature of a human may rise due to poor physical condition. Thus, the body temperature may rise without alcohol when the exhalation sensing unit 10 does not sense alcohol and the temperature acquired by the temperature acquisition unit 484 is equal to or greater than the body temperature threshold. Thus, in such a case, the notification unit 20 notifies the operator 470 of a minor warning. The minor warning is a notification, such as “high body temperature. If you are drunk, please stop the car,” for example.

The moving state sensing unit 488 senses a moving state of the moving object 400. The moving state sensing unit 488 is a speed sensor, an acceleration sensor, or an angular velocity sensor, for example. The moving state sensing unit 488 may sense whether the moving object 400 is moving or is stopped. The moving state sensing unit 488 may sense that the moving object 400 is moving when the moving object 400 is moving at a constant speed or is accelerating or decelerating. The moving state sensing unit 488 may be a location sensor, and may acquire a change in time in the location information, to sense whether the moving object 400 is moving or is stopped.

The information acquisition unit 70 of the exhalation sensing apparatus 300 acquires the moving state sensed by the moving state sensing unit 488 of the moving object 400. The information acquisition unit 70 may acquire the moving state wirelessly. The notification unit 20 may notify the operator 470 of an instruction based on the moving state sensed by the moving state sensing unit 488 and the sensing result of alcohol. For example, when the moving state is moving and alcohol is sensed, the notification unit 20 notifies the operator 470 of a message such as “There is a possibility of drunk driving. Please stop the car immediately”, “For switching into drive assistance mode, please remove your hand from the handle”. The notification unit 20 may notify the exhalation alcohol concentration with a graphic. The notification unit 20 may notify information for facilitating accurate inspection. The information for facilitating accurate inspection is information such as “there is a possibility of drunk driving. Please put your face closer and blow your breath”, for example.

The notification unit 20 may not notify anything when the moving state is moving and alcohol is not sensed. When the moving state is stopped and alcohol is sensed, the notification unit 20 may notify a message such as “please do not drive now. Please wait for X hours”. X hours is an expected time for reaching the exhalation alcohol concentration that does not correspond to drunk driving, for example. The notification unit 20 may not notify anything when the moving state is stopped and alcohol is not sensed.

Opening and closing information acquisition unit 490 acquires opening and closing information of the window 450. In a case of a power window type that is closed by moving the window 450 from downward to upward, a sensor that senses a contact between an upper edge of the window 450 and a mobile body housing 410 may be provided on a part of the mobile body housing 410, at which the upper edge of the window 450 and the mobile body housing 410 is in contact with each other. The opening and closing information acquisition unit 490 may acquire information that indicates that the window 450 is in a closed state when the sensor senses a contact, and may acquire information that indicates that the window 450 is in an open state when the sensor does not sense a contact. In addition, the opening and closing information acquisition unit 490 may acquire opening and closing information of the door 460.

The opening and closing information acquisition unit 490 may acquire the opening and closing information of the window 450, based on a ratio of the area of the window 450 that is exposed to the operator's cabin 440 relative to the area that is enclosed with a frame portion of the window 450. The ratio may have a predetermined threshold. The threshold is 80%, for example. When the ratio is 100%, the window 450 is in a completely closed state. When the ratio is 0%, the window 450 is in a completely open state. When the ratio is equal to or greater than the threshold and less than 100%, the window 450 is not in a completely closed state, but the opening and closing information acquisition unit 490 may acquire the state as a closed state.

The information acquisition unit 70 of the exhalation sensing apparatus 300 acquires opening and closing information acquired by the opening and closing information acquisition unit 490 of the moving object 400. The information acquisition unit 70 may acquire the opening and closing information wirelessly. The notification unit 20 may notify the operator 470 of an instruction related to an operation of the window 450 or the door 460, based on the sensing result of exhalation and the opening and closing information. For example, when the exhalation sensing unit 10 does not sense the exhalation and the opening and closing information is opened, the notification unit 20 notifies the operator 470 of an instruction that facilitates closing the window 450 or the door 460. When the window 450 or the door 460 is in an open state, the outside air may be introduced into the operator's cabin 440. Thus, there is a possibility that the exhalation sensing unit 10 cannot sense the exhalation due to the outside air introduced into the operator's cabin 440. Thus, the notification unit 20 notifies the operator 470 of an instruction that facilitates occupying the window 450. Thereby, the exhalation sensing unit 10 easily senses the presence or absence of exhalation accurately.

The exhalation sensing unit 10 may sense a gas of organic matter other than alcohol. In a case of an NDIR type sensor, the wavelength of infrared being absorbed depends on the type of gas. Accordingly, when the carbon dioxide concentration measurement unit 120 of the component sensing unit 110 (see FIG. 4) is a non-dispersive infrared absorption (NDIR type) sensor, the carbon dioxide concentration measurement unit 120 can measure the concentration of a gas other than carbon dioxide. The carbon dioxide concentration measurement unit 120 may sense a gas of organic matter other than alcohol. A gas of organic matter other than alcohol is a volatile gas such as benzene, a volatile gas and the like of gasoline, a gas and the like of a perfuming agent for a car, for example.

The notification unit 20 may notify the operator 470 of an instruction based on the concentration of the gas of the organic matter. The notification unit 20 may change the instruction for the operator 470 based on the type of gas of the organic matter. The notification unit 20 may differentiate an instruction in a case where the gas of the organic matter is a type of high risk such as an explosion and an instruction in a case where the gas is a type of low risk. In the storage unit 50, a threshold of the concentration in the gas of a type of high risk may be pre-stored for each gas type.

When a gas sensed by the exhalation sensing unit 10 is a gas of an organic matter, the exhalation sensing unit 10 may sense a gas of organic matter included in the air of the operator's cabin 440. When the window 450 is in an open state and the moving object 400 is stopped at a gasoline stand, for example, volatilized gasoline may be introduced into the operator's cabin 440. In such a case, the exhalation sensing unit 10 may sense volatilized gasoline.

The determination unit 80 may determine the magnitude of the gas concentration of the organic matter sensed by the exhalation sensing unit 10 and a threshold of the gas concentration of the organic matter. When the gas of the organic matter is a volatile gas of gasoline, and the gas concentration of the organic matter sensed by the exhalation sensing unit 10 is equal to or greater than the threshold, the notification unit 20 may notify a message such as “Danger. There may be full of gasoline”. When the gas of the organic matter is a gas of the type of low risk and the concentration of the gas of the type that is sensed by the exhalation sensing unit 10 is equal to or greater than the threshold, the notification unit 20 may notify a message such as please perform ventilation of operator's cabin 440”.

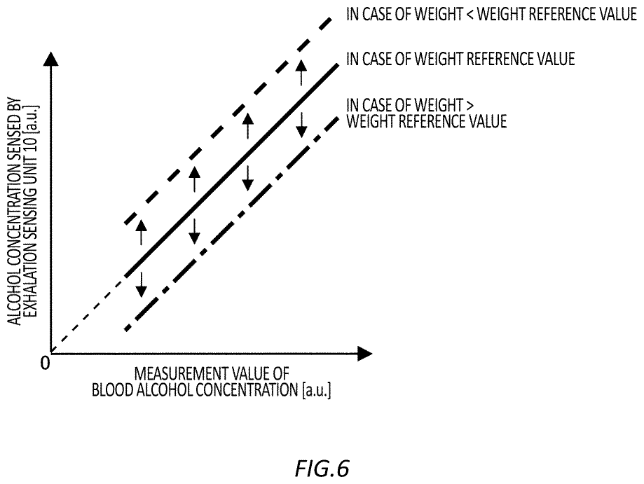

FIG. 6 illustrates an example of a relationship between a measurement value of a blood alcohol concentration and an alcohol concentration measured by the exhalation sensing unit 10. The weight acquisition unit 482 acquires the weight of the operator 470. When the operator 470 is a human, the weight acquisition unit 482 acquires the weight of the human. When the operator 470 is a human, the weight reference value shown in FIG. 6 may be an average value of the weight of a human. The average value is 60 kg, for example. A case of a weight reference value shown in a solid line in FIG. 6 indicates a relationship between a measurement value of the blood alcohol concentration and the alcohol concentration measured by the exhalation sensing unit 10 in a case where the weight is of an average value of a human, for example.

When the operator 470 is a human, the percentage of blood weight of a human for the weight of a human is approximately constant regardless of the weight. The percentage is, for example, 7%. Accordingly, when the same amount of alcohol is taken, the greater the weight of a human is, the easier the blood alcohol concentration decreases, and the less the weight of a human is, the easier the blood alcohol concentration increases. Accordingly, as shown in FIG. 6, when the weight is greater than the weight reference value, a relationship between a measurement value of the blood alcohol concentration and the alcohol concentration measured by the exhalation sensing unit 10 easily becomes a relationship indicated with one dot chain line, and when the weight is less than the weight reference value, the relationship easily becomes a relationship indicated with a coarse dashed line.

In the storage unit 50, a level of divergence between the solid line and one dot chain line, and a level of divergence between the solid line and the coarse dashed line shown in FIG. 6 may be pre-stored for each weight. The level of divergence may be the difference between the measurement value of the blood alcohol concentration and the alcohol concentration sensed by the exhalation sensing unit 10.

The information acquisition unit 70 of the exhalation sensing apparatus 300 acquires the weight acquired by the weight acquisition unit 482 of the moving object 400. The information acquisition unit 70 may acquire the weight wirelessly. The exhalation sensing unit 10 may correct the alcohol concentration sensed by the exhalation sensing unit 10, based on the weight acquired by the weight acquisition unit 482. For example, the exhalation sensing unit 10 acquires the corrected alcohol concentration by integrating a level of divergence for each weight stored in the storage unit 50 on the alcohol concentration sensed by the exhalation sensing unit 10. The level of divergence is a level of divergence corresponding to the weight acquired by the weight acquisition unit 482.

The notification unit 20 may notify the operator 470 of an instruction based on the alcohol concentration corrected by the exhalation sensing unit 10. Thereby, the notification unit 20 can notify an instruction based on an alcohol concentration that is more accurate, which is obtained by reflecting the weight of the operator 470.

The image capturing unit 486 captures an image of the operator 470. The information acquisition unit 70 of the exhalation sensing apparatus 300 acquires the image captured by the image capturing unit 486 of the moving object 400. The information acquisition unit 70 may acquire the image wirelessly. The determination unit 80 of the exhalation sensing apparatus 300 determines the gender of the operator 470 based on the image acquired by the information acquisition unit 70.

In general, the alcohol decomposition rate of a woman is often slower than the alcohol decomposition rate of a man. Thus, when the same amount of alcohol is taken, the blood alcohol concentration in the body of a woman easily increases than the blood alcohol concentration in the body of a man. Accordingly, a relationship between a measurement value of the blood alcohol concentration and the alcohol concentration measured by the exhalation sensing unit 10 shown in FIG. 6 may be different for each gender.

In the storage unit 50, a level of divergence between the solid line and one dot chain line, and a level of divergence between the solid line and the coarse dashed line shown in FIG. 6 may be pre-stored for each weight and for each gender.

The exhalation sensing unit 10 may correct the alcohol concentration sensed by the exhalation sensing unit 10, based on the weight acquired by the weight acquisition unit 482 and the image captured by the image capturing unit 486. Exhalation sensing unit 10 may correct the alcohol concentration based on the weight acquired by the weight acquisition unit 482 and the gender determined by the determination unit 80. For example, the exhalation sensing unit 10 acquires the corrected alcohol concentration by integrating a level of divergence for each weight and for each gender stored in the storage unit 50 on the alcohol concentration sensed by the exhalation sensing unit 10. The level of divergence is a level of divergence corresponding to the weight acquired by the weight acquisition unit 482 and the gender determined by the determination unit 80. The notification unit 20 may notify the operator 470 of an instruction based on the alcohol concentration corrected by the exhalation sensing unit 10.

The determination unit 80 may determine whether or not the operator 470 is wearing a mask based on the image captured by the image capturing unit 486. The notification unit 20 may notify the operator 470 of an instruction based on the image captured by the image capturing unit 486. For example, the notification unit 20 notifies a message such as “please remove your mask and blow your breath.” when the determination unit 80 determines that the operator 470 is wearing a mask.

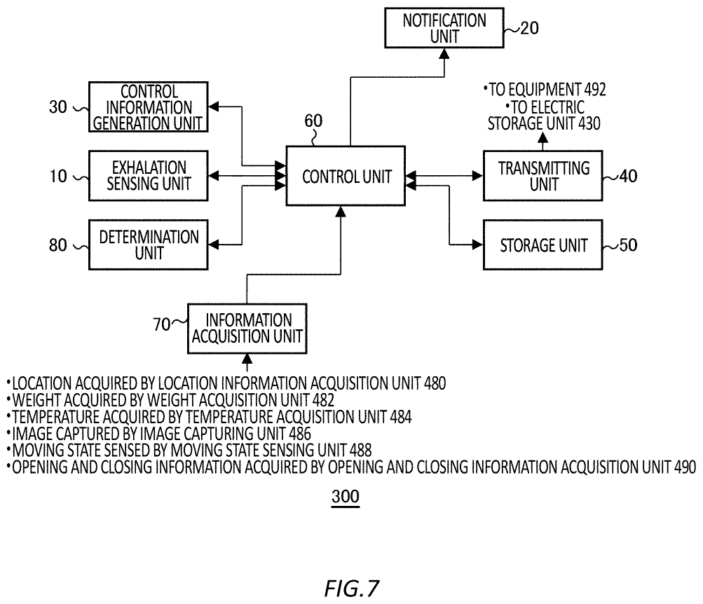

FIG. 7 is a block diagram illustrating another example of the exhalation sensing apparatus 300 according to one embodiment of the present invention. The exhalation sensing apparatus 300 of the present example is different from the exhalation sensing apparatus 300 in FIG. 2 in that the control information generation unit 30 and the transmitting unit 40 is further included.

The control information generation unit 30 generates control information for controlling the equipment 492 included in the moving object 400 (see FIG. 5) based on the sensing result of the exhalation obtained by the exhalation sensing unit 10. The equipment 492 may be equipment related to the controlling operation of the moving object 400. The equipment related to the operation is, for example, a power portion 420 (see FIG. 1), a braking unit, a steering unit, and the like. The equipment 492 may be electronic equipment that includes the moving object 400. The electronic equipment is a car navigation, a car audio, a power window, adjustment equipment of a driving position, and the like, for example. The transmitting unit 40 transmits the control information to the equipment 492. The transmitting unit 40 may transmit the control information to the electric storage unit 430. Thereby, the exhalation sensing apparatus 300 can control the equipment 492 based on the sensing result of the exhalation.

In the storage unit 50, the sensing result of the exhalation may be stored in association with a relationship between control information corresponding to the sensing result and equipment 492 corresponding to the control information and the contents of the control. The control information generation unit 30 may generate the control information of at least one of a plurality of pieces of control information based on the sensing result of the exhalation or the relationship stored in the storage unit 50. The control information generation unit 30 may generate a plurality of pieces of control information different from each other, based on a plurality of different combinations of the interval between exhalations and the speed of change in the exhalation amount. The transmitting unit 40 may transmit the control information and the content of control to the equipment 492 corresponding to the control information, based on said at least one control information and the relationship of the control information stored in the storage unit 50 and the equipment 492.

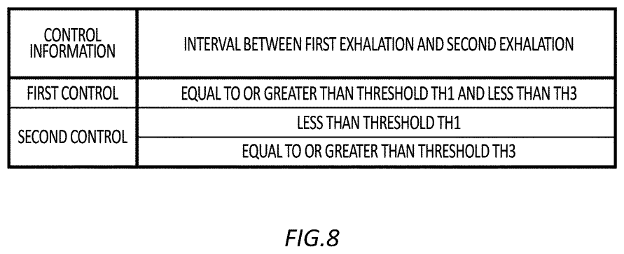

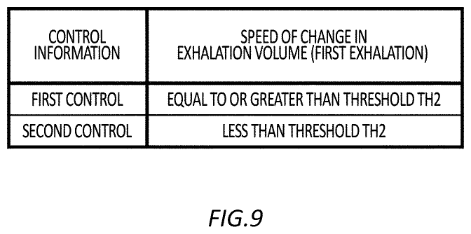

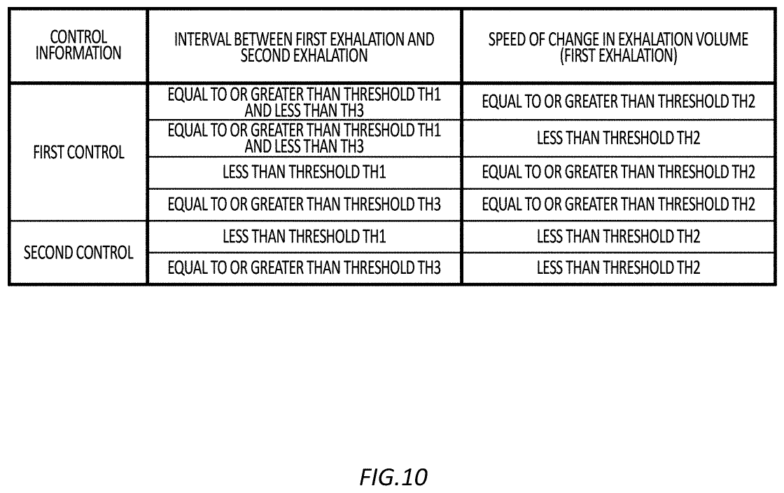

FIG. 8 illustrates an example of the correspondence between an interval between exhalations and control information, FIG. 9 illustrates an example of the correspondence between the speed of change in the exhalation volume and control information, and FIG. 10 illustrates an example of the correspondence between the interval between exhalations and the speed of change in the exhalation volume and control information. Herein, the interval between exhalations refers to the time interval from the first sensed exhalation to the second sensed exhalation. The mean for sensing the exhalation may be a method for measuring the CO₂ concentration or the flow rate. For example, the mean for sensing the exhalation includes an optical gas sensor or the like, but not limited thereto. In addition, the threshold for recognizing the exhalation may be a case in which the CO₂ concentration or the flow rate exceeds a predetermined value. The speed of change in the exhalation volume refers to the change in the exhalation volume per unit time. The mean for sensing the change in the exhalation volume may be a method for measuring the CO₂ concentration at a predetermined time interval to calculate its changed amount. Alternatively, the flow rate of exhalation that leaves the body per unit time may be measured. As long as the mean is an approach can sense the change in the exhalation volume per unit time, it is not particularly limited.

A threshold of the interval between exhalations is referred to as threshold th1, and threshold th3. The threshold th1 may be a lower limit value of the interval between of exhalations of a human in a normal state. The threshold th1 may be an upper limit value of the interval between of exhalations of a human in a normal state. When the interval between exhalations is less than the threshold th1, it is determined that a human is consciously reducing the interval between exhalations. When the interval between exhalations is equal to or greater than the threshold th3, it is determined that a human is consciously increasing the interval between exhalations. Each threshold in the present specification may be preset by a manufacturer of the exhalation sensing apparatus 300 and the like based on statistical data or another data. A threshold of the speed of change in the exhalation volume is referred to as threshold th2. The threshold th2 may be the speed of change in the exhalation volume at the time of breathing of a normal human. When the speed of change in the exhalation volume is less than the threshold th2, it is determined that the human is weakening the exhalation consciously.

The storage unit 50 may store the interval between exhalations, the speed of change in the exhalation volume, and the control information in association with each other. The control information may be associated with the equipment 492 corresponding to the control information. The first exhalation and the second exhalation shown in FIG. 8 refer to two successive exhalations.

The first control is a control in a case in which the interval between exhalations is equal to or greater than the threshold th1 and less than the threshold th3 (FIG. 8), or in which the speed of change of the exhalation volume is equal to or greater than the threshold th2 (FIG. 9). When the interval between exhalations is equal to or greater than the threshold th1 and less than the threshold th3, the human has a high probability of breathing normally. When the speed of change of the exhalation volume is equal to or greater than the threshold th2, the human has a high probability of breathing normally, or there is a high probability of blowing a sufficient volume of exhalation required for alcohol sensing. Accordingly, it is determined to be a state in which the alcohol sensing can be correctly performed. In addition, in the equipment control performed by the exhalation, it is determined that a special operation is not required. Thus, the first control may be control information indicating that an operation related to the failure of alcohol sensing is not performed, and no equipment 492 is controlled. Herein, an example of the operation related to the failure of alcohol sensing includes notifying that the measurement is not correctly performed, performing a re-measurement, issuing a notification for facilitating an accurate re-measurement, issuing a notification for facilitating stopping the car when the car is driving, reducing the speed, stopping the engine, transmitting outside that the measurement is not correctly performed, or the like. Herein, an example of notifications for facilitating the accurate re-measurement includes an instruction such as putting the face closer, blowing the breath to a predetermined place, closing the window, stopping the air conditioner, changing the orientation of blowing, or the like.

For further preference, the first control may be a control that meets at least one of a case in which the interval between exhalations is equal to or greater than the threshold th1 and less than th3, or a case in which the speed of change of the exhalation volume is equal to or greater than the threshold th2 (FIG. 10). By using a combination of pieces of information of the interval between exhalations and the speed of change of the exhalation volume, it can be less susceptible to disturbances. Herein, an example of the disturbance includes air conditioning, wind entering from the window, exhalation of a passenger, a sanitization product, a perfuming agent, or the like. The second control is a case in which the state of the first control is not met at the time of the alcohol sensing. In this case, it may be determined that the alcohol sensing is not correctly performed. In this case, the second control may be control information for performing the operation related to the failure of the foregoing alcohol sensing. Due to the configuration described above, a situation in which driving continues regardless of an unauthorized sensing result can be avoided by determining that the alcohol sensing is not successfully performed when the breathing is not normal, to control each equipment.

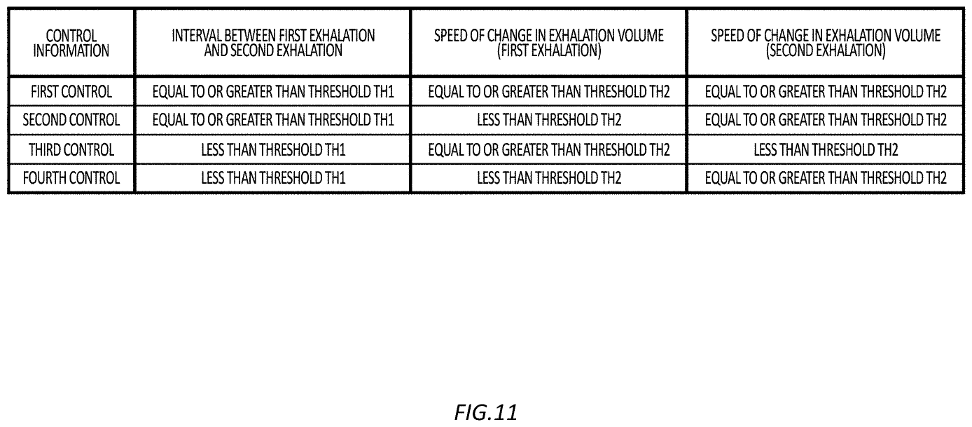

In addition, the following another embodiment may be employed by utilizing the speed of change of the second exhalation volume (FIG. 11). The first control is a case in which the interval between exhalations is equal to or greater than the threshold th1 and the speed of change of the exhalation volume in the first exhalation is equal to or greater than the threshold th2, and the speed of change of the exhalation volume in the second exhalation is equal to or greater than the threshold th2. In this case, a human has a high probability of normal breathing.

The second control is a case in which the interval between exhalations is equal to or greater than the threshold th1 and the speed of change of the exhalation volume in the first exhalation is less than the threshold th2, and the speed of change of the exhalation volume in the second exhalation is equal to or greater than the threshold th2. When the interval between exhalations is less than the threshold th2, a human has a high probability of consciously blowing a strong exhalation to the inlet 102. Accordingly, the second control may be a case in which a strong exhalation is blown once by a human with a normal speed of exhalation. The second control may be control information indicating that a first equipment 492 among a plurality of pieces of equipment 492 is controlled. For example, the second control is control information indicating that a car navigation will be activated.

The third control is a case in which the interval between exhalations is less than the threshold th1 and the speed of change of the exhalation volume in the first exhalation is equal to or greater than the threshold th2, and the speed of change of the exhalation volume in the second exhalation is less than the threshold th2. The third control may be a case in which a human blows a strong exhalation first and then blows a weak exhalation for the second time, at a fast interval between the exhalations. The third control may be control information indicating that a second equipment 492 among a plurality of pieces of equipment 492 is controlled. For example, the third control is control information indicating that a car audio will be activated.

The fourth control is a case in which the interval between exhalations is less than the threshold th1 and the speed of change of the exhalation volume in the first exhalation is less than the threshold th2, and the speed of change of the exhalation volume in the second exhalation is equal to or greater than the threshold th2. The fourth control may be a case in which a human blows a weak exhalation first and then blows strong exhalation for the second time, at a fast interval between the exhalations. The fourth control may be control information indicating that a third equipment 492 among a plurality of pieces of equipment 492 is controlled. For example, the fourth control is control information indicating that the air conditioning in the operator's cabin 440 (see FIG. 1) will be activated. With the embodiment described above, by breathing that is not normal, the equipment can be operated with only breathing without using the hands.

The exhalation sensing unit 10 may sense the interval between exhalations and may acquire the speed of change in the amount of exhalation. For example, the exhalation sensing unit 10 may sense whether the interval between exhalations is equal to or greater than the threshold th1 or less than the threshold th1, and may acquire whether the speed of change in the exhalation volume is equal to or greater than the threshold th2 or less than the threshold th2. The control information generation unit 30 may generate a plurality of pieces of control information different from each other based on the interval between exhalations and the speed of change in the exhalation volume. In the example of FIG. 11, the control information generation unit 30 generates four pieces of control information different from each other, based on whether the interval between exhalations is equal to or greater than the threshold th1 or less than the threshold th1, and whether the speed of change in the exhalation volume is equal to or greater than the threshold th2 or less than the threshold th2. The control information may be a single piece or multiple pieces, and the quantity thereof may be set to be any number.

After the transmitting unit 40 transmits the control information to the equipment 492, the notification unit 20 may further notify additional information for controlling the equipment 492 based on another sensing result of the exhalation. Another sensing result of the exhalation refers to exhalation later than the second exhalation in the example of FIG. 11. For example, when a car audio is activated by the third control in the example of FIG. 11, additional information for turning up or turning down the sound volume of the car audio is notified based on the sensing result of the third exhalation. The additional information for turning up the sound volume of the car audio is a message such as “if you would like to turn up the sound volume, please continue to blow the breath for 2 seconds”, for example. The additional information for turning down the sound volume is similar.

The control information may be information for controlling the power portion 420. The control information may be information for activating the power portion 420 or information for stopping the power portion 420. For example, when the exhalation volume is equal to or greater than the threshold, the transmitting unit 40 transmits the control information for activating the power portion 420 to the power portion 420. When the exhalation volume is less than the threshold, the transmitting unit 40 transmits the control information for stopping the power portion 420 to the power portion 420.

When the exhalation sensing unit 10 senses alcohol included in the exhalation and the determination unit 80 determines that the alcohol concentration is equal to or greater than the threshold, the control information generation unit 30 may generate control information for stopping the power portion 420. The transmitting unit 40 may transmit the control information to the power portion 420. Thereby, drunk driving by the operator 470 may be prevented.

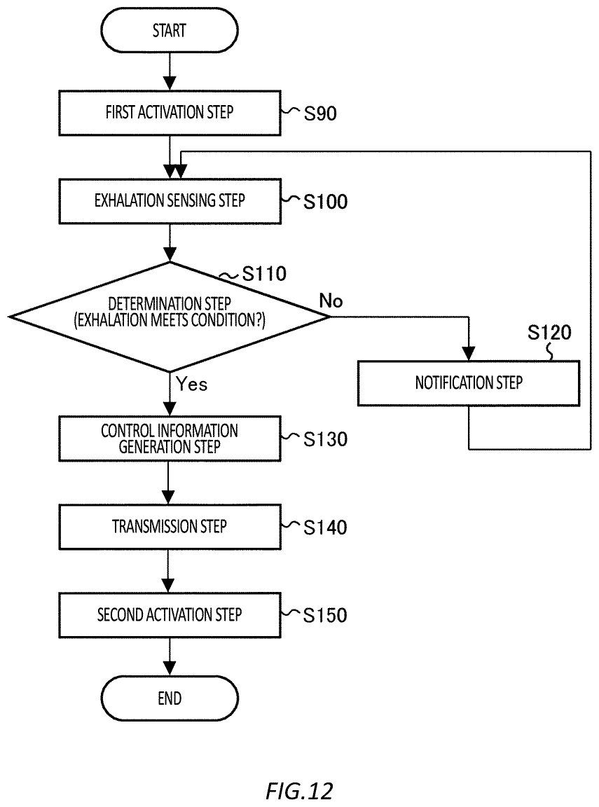

FIG. 12 is a flowchart illustrating an example of an exhalation sensing method according to one embodiment of the present invention. The exhalation sensing method of the present example describes an example of the exhalation sensing apparatus 300 shown in FIG. 7. The exhalation sensing method includes an exhalation sensing step S100 and a notification step S120. The exhalation sensing method may include a first activation step S90, a determination step S110, a control information generation step S130, a transmission step S140, and a second activation step S150.

The first activation step S90 is a step at which the electric storage unit 430 (see FIG. 1) starts to supply electrical power to the exhalation sensing apparatus 300. The first activation step S90 may be a step at which the electric storage unit 430 activates the exhalation sensing unit 10 and the notification unit 20 by starting the supply of electrical power to the exhalation sensing apparatus 300.

The exhalation sensing step S100 is a step at which the exhalation sensing unit 10 senses the exhalation of the operator 470 in a state in which the operator 470 of the moving object 400 is not in contact therewith. The exhalation sensing step S100 is a step at which the exhalation sensing unit 10 senses the exhalation before the notification unit 20 notifies an instruction. The exhalation sensing step S100 may be a step at which the exhalation sensing unit 10 senses alcohol included in the exhalation.

The determination step S110 is a step at which the determination unit 80 determines whether the exhalation sensed in the exhalation sensing step S100 meets a condition for controlling the equipment 492. The determination step S110 is a step at which whether the exhalation sensed in the exhalation sensing step S100 corresponds to any case from the first control to the fourth control shown in FIG. 8 to FIG. 11, for example, is determined. When it is determined that the exhalation meets the condition for controlling the equipment 492 in the determination step S110, the exhalation sensing method proceeds to the control information generation step S130. When it is determined that the exhalation does not meet the condition for controlling the equipment 492 in the determination step S110, the exhalation sensing method proceeds to the notification step S120.

The notification step S120 is a step at which the notification unit 20 notifies the operator 470 of an instruction. The notification step S120 is a step at which the notification unit 20 notifies the operator 470 of an instruction, based on the sensing result of exhalation obtained by the exhalation sensing unit 10.

The control information generation step S130 is a step at which the control information generation unit 30 generates control information for controlling the equipment 492 based on the sensing result of exhalation in the exhalation sensing step S100. In the examples of FIG. 8 from FIG. 11, the control information generation step S130 is a step at which the control information generation unit 30 generates control information according to control among at least one of the first control to the fourth control based on the sensing result of exhalation. The transmission step S140 is a step at which the transmitting unit 40 transmits the control information generated in the control information generation step S130 to the equipment 492. The transmission step S140 may be a step at which the transmitting unit 40 transmits the control information to the electric storage unit 430. The second activation step S150 is a step at which a control unit provided in the moving object 400 activates the equipment 492 based on the control information transmitted in the transmission step S140. The second activation step S150 may be a step at which the control unit provided in the moving object 400 activates the power portion 420 based on the transmitted control information.

The calibration information generation unit 140, the result correction unit 150 and the control information generation unit 30 described in FIG. 1 to FIG. 12 may be achieved by installing a program in one computer or a plurality of computers. These programs may be recorded in a computer-readable medium.

Various embodiments of the present invention may be described with reference to flowcharts and block diagrams whose blocks may represent (1) steps of processes in which operations are performed or (2) sections of apparatuses responsible for performing operations. Certain stages and sections may be implemented by a dedicated circuit, a programmable circuit supplied together with a computer-readable instruction stored on a computer-readable medium, and/or processors supplied together with the computer-readable instruction stored on the computer-readable medium. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer-readable medium may include any tangible device that can store instructions to be executed by a suitable device, and as a result, the computer-readable medium having instructions stored thereon includes a product including instructions that can be executed in order to create means for executing operations designated in the flowcharts or block diagrams. Examples of the computer-readable medium may include an electronic storage medium, a magnetic storage medium, an optical storage medium, an electromagnetic storage medium, a semiconductor storage medium, and the like. More specific examples of the computer-readable medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a Blu-ray (registered trademark) disk, a memory stick, an integrated circuit card, or the like.

The computer-readable instruction may include: an assembler instruction, an instruction-set-architecture (ISA) instruction; a machine instruction; a machine dependent instruction; a microcode; a firmware instruction; state-setting data; or either a source code or an object code described in any combination of one or more programming languages, including an object oriented programming language such as Smalltalk (registered trademark), JAVA (registered trademark), C++, or the like, and a conventional procedural programming language such as a “C” programming language or a similar programming language.

The computer-readable instructions may be provided for a processor or programmable circuit of a general purpose computer, special purpose computer, or other programmable data processing apparatuses such as a computer locally or via a wide area network (WAN) such as a local area network (LAN), the Internet, or the like, and execute the computer-readable instructions in order to create means for executing the operations designated in flowcharts or block diagrams. Herein, the computer may be a PC (personal computer), a tablet computer, a smartphone, a workstation, a server computer, a general purpose computer, a special purpose computer, or the like, or may be a computer system to which a plurality of computers are connected. Such computer system to which the plurality of computers are connected is also referred to as a distributed computing system, and is a computer in a broad sense. In the distributed computing system, the plurality of computers collectively execute the program by each of the plurality of computers performing a part of the program and passing the data during program execution between the computers as needed.

Examples of the processor include a computer processor, a central processing unit (CPU), a processing unit, a microprocessor, a digital signal processor, a controller, a microcontroller, and the like. The computer may include one processor or a plurality of processors. In a multi-processor system including a plurality of processors, the plurality of processors collectively execute a program by each of the processors executing a portion of the program, and passing data during the execution of the program among the processors as needed. For example, in execution of multiple tasks, each of the plurality of processors may execute a portion of each task pieces by pieces by performing task-switching for each time slice. In this case, which portion of one program each processor is responsible for executing dynamically changes. Moreover, which portion of the program each of the plurality of processors is responsible for executing may be determined statically by multiprocessor-aware programming.

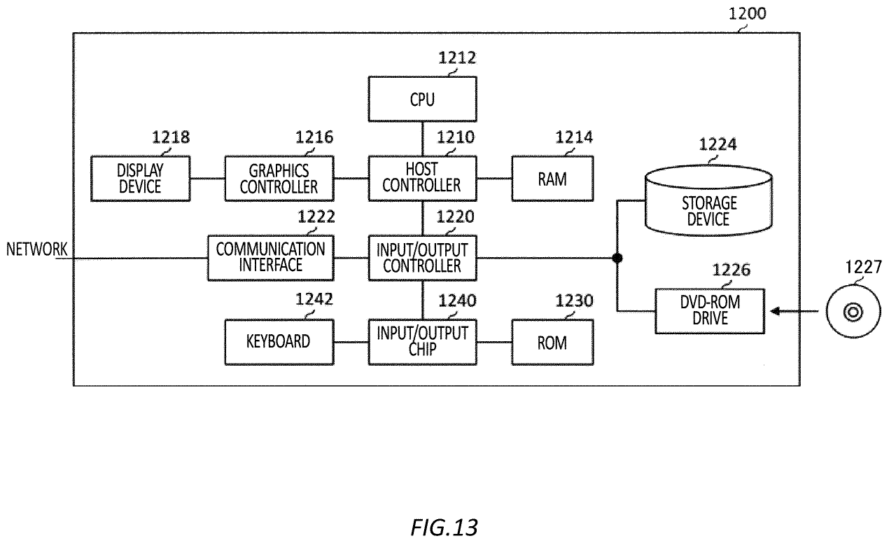

FIG. 13 illustrates an example of a computer 1200 in which a plurality of aspects of the present invention may be entirely or partially embodied. A program that is installed in the computer 1200 can cause the computer 1200 to function as an operation associated with an apparatus associated with the embodiment of the present invention or one or more sections of the apparatus, or cause the computer 1200 to perform the operation or the one or more sections thereof, and/or cause the computer 1200 to perform processes of the embodiment of the present invention or steps thereof. Such a program may be performed by a CPU 1212 so as to cause the computer 1200 to perform certain operations associated with some or all of the blocks of flowcharts and block diagrams described herein.

The computer 1200 in accordance with the present embodiment includes a CPU 1212, a RAM 1214, a graphic controller 1216, and a display device 1218, which are mutually connected by a host controller 1210. The computer 1200 also includes input/output units such as a communication interface 1222, a storage device 1224 such as a hard disk drive, a DVD-ROM drive 1226 and an IC card drive, which are connected to the host controller 1210 via an input/output controller 1220. The computer also includes input/output units of a legacy such as a ROM 1230 and a keyboard 1242, which are connected to the input/output controller 1220 via an input/output chip 1240.

The CPU 1212 operates according to programs stored in the ROM 1230 and the RAM 1214, thereby controlling each unit. The graphic controller 1216 obtains image data generated by the CPU 1212 on a frame buffer or the like provided in the RAM 1214 or in itself, and causes the image data to be displayed on a display device 1218.

The communication interface 1222 communicates with other electronic devices via a network. The storage device 1224 stores the program and data used by the CPU 1212 within the computer 1200. The DVD-ROM drive 1226 reads the program or data from the DVD-ROM 1227 and provides the program or data to the storage device 1224 via the RAM 1214. The IC card drive reads the programs and the data from the IC card, and/or writes the programs and the data to the IC card.

The ROM 1230 stores therein a boot program or the like that is performed by the computer 1200 at the time of activation, and/or a program depending on the hardware of the computer 1200. The input/output chip 1240 may also connect various input/output units to the input/output controller 1220 via a parallel port, a serial port, a keyboard port, a mouse port, and the like.

Programs are provided by a computer-readable medium such as the DVD-ROM 1227 or the IC card. The program is read from the computer-readable medium, is installed on a storage device 1224, a RAM 1214, or a ROM 1230, which are an example of a computer-readable medium, and is executed by the CPU 1212. Information processing written in these programs is read by the computer 1200, and provides cooperation between the programs and the various types of hardware resources described above. An apparatus or method may be constituted by realizing the operation or processing of information in accordance with the usage of the computer 1200.

For example, when communication is performed between the computer 1200 and an external device, the CPU 1212 may perform a communication program loaded onto the RAM 1214 to instruct communication processing to the communication interface 1222, based on the processing described in the communication program. The communication interface 1222 reads the transmission data stored in a transmission buffer processing region provided on a recording medium such as a RAM 1214, a storage device 1224, a DVD-ROM 1227, or an IC card under the control of a CPU 1212, transmits the read transmission data to the network, or writes the reception data received from the network to a reception buffer processing region or the like provided on a recording medium.

The CPU 1212 may cause all or a needed portion of the file or database stored in an external recording medium such as a storage device 1224, a DVD-ROM drive 1226 (a DVD-ROM 1227), an IC card, or the like to be read to the RAM 1214 and perform various types of processing on the data on the RAM 1214. The CPU 1212 may then write back the processed data to the external recording medium.

Various types of information such as various types of programs, data, tables, and databases may be stored in a recording medium and subjected to information processing. The CPU 1212 may perform various types of processing on the data read from the RAM 1214, which includes various types of operations, information processing, condition judging, conditional branch, unconditional branch, search/replace of information, etc., as described throughout this disclosure and designated by an instruction sequence of programs, and writes the result back to the RAM 1214. In addition, the CPU 1212 may search for information in a file, a database, or the like in the recording medium. For example, when a plurality of entries, each having an attribute value of a first attribute associated with an attribute value of a second attribute, are stored in the recording medium, the CPU 1212 may retrieve, out of the plurality of entries, an entry with the attribute value of the first attribute specified that meets a condition, read the attribute value of the second attribute stored in said entry, and thereby acquiring the attribute value of the second attribute associated with the first attribute satisfying a predetermined condition.

The above-explained program or software modules may be stored in the computer-readable medium on or near the computer 1200. In addition, a recording medium such as a hard disk or a RAM provided in a server system connected to a dedicated communication network or the Internet can be used as the computer-readable medium, thereby providing the program to the computer 1200 via the network.

While the present invention has been described by way of the embodiments, the technical scope of the present invention is not limited to the above-described embodiments. It is apparent to persons skilled in the art that various alterations or improvements can be made to the above described embodiments. It is also apparent from description of the claims that the embodiments to which such alterations or improvements are made may be included in the technical scope of the present invention.

It should be noted that each process of the operations, procedures, steps, stages, and the like performed by the apparatus, system, program, and method shown in the claims, specification, or drawings can be executed in any order as long as the order is not indicated by “prior to”, “before”, or the like and as long as the output from a previous process is not used in a later process. Even if the operation flow is described using phrases such as “first” or “next” for the sake of convenience in the claims, specification, or drawings, it does not necessarily mean that the process must be performed in this order.

What is claimed is:

1. An exhalation sensing apparatus provided in an operator's cabin of a moving object, the exhalation sensing apparatus comprising:
an exhalation sensing unit that senses an exhalation in a state in which an operator of the moving object is not in contact therewith; and
a notification unit that notifies the operator of an instruction, wherein
the exhalation sensing unit senses the exhalation before the notification unit notifies of the instruction, and
the notification unit notifies the operator of the instruction based on a sensing result of the exhalation obtained by the exhalation sensing unit.

2. The exhalation sensing apparatus according to claim 1, wherein
the exhalation sensing unit is contained in a sensing housing fixed to the moving object, and
the sensing housing has an inlet through which the exhalation passes.

3. The exhalation sensing apparatus according to claim 2, wherein
the exhalation introduces the sensing housing for a certain time period through the inlet, and
the exhalation sensing unit senses an exhalation based on a carbon dioxide concentration in an interior space of the sensing housing.

4. The exhalation sensing apparatus according to claim 1, wherein
the exhalation sensing unit is contained in a sensing housing, and
the sensing housing has an air blowing unit that delivers air of the operator's cabin to the exhalation sensing unit.

5. The exhalation sensing apparatus according to claim 1, wherein
the exhalation sensing unit senses alcohol included in the exhalation, and
the notification unit notifies the operator of the instruction based on a sensing result of alcohol in the exhalation.

6. The exhalation sensing apparatus according to claim 5, wherein
the moving object has a location information acquisition unit that acquires a location of the moving object,
the exhalation sensing unit determines a magnitude relationship between an alcohol concentration in the exhalation and a reference value of an alcohol concentration in the location, and
the notification unit notifies the operator of the instruction based on the magnitude relationship between the alcohol concentration in the exhalation and the reference value.

7. The exhalation sensing apparatus according to claim 5, wherein
the moving object has a weight acquisition unit that acquires a weight of the operator,
the exhalation sensing unit corrects the alcohol concentration based on the weight, and
the notification unit notifies the operator of the instruction based on the alcohol concentration that is corrected.

8. The exhalation sensing apparatus according to claim 7, wherein
the moving object has an image capturing unit that captures an image of the operator,
the exhalation sensing unit corrects the alcohol concentration based on the weight and the image captured by the image capturing unit, and
the notification unit notifies the operator of the instruction based on the alcohol concentration that is corrected.

9. The exhalation sensing apparatus according to claim 5, wherein
the moving object has a temperature acquisition unit that acquires a temperature of the operator, and
the notification unit notifies the operator of the instruction based on the sensing result of the alcohol and the temperature.

10. The exhalation sensing apparatus according to claim 5, wherein
the moving object has a moving state sensing unit that senses a moving state of the moving object, and
the notification unit notifies the operator of the instruction based on the moving state and the sensing result of the alcohol.

11. The exhalation sensing apparatus according to claim 1, wherein
the exhalation sensing unit senses a gas of organic matter other than alcohol, and
the notification unit notifies the instruction based on a concentration of the gas of the organic matter.

12. The exhalation sensing apparatus according to claim 1, wherein
the operator's cabin has a window that is openable and closable,
the moving object has an opening and closing information acquisition unit that acquires opening and closing information of the window, and
the notification unit notifies the operator of an instruction related to an operation of the window, based on the sensing result of the exhalation and the opening and closing information.

13. The exhalation sensing apparatus according to claim 1, further comprising:
a control information generation unit that generates control information for controlling equipment included in the moving object based on the sensing result of the exhalation obtained by the exhalation sensing unit; and
a transmitting unit that transmits the control information to the equipment.

14. The exhalation sensing apparatus according to claim 13, wherein after the transmitting unit transmitted the control information to the equipment, the notification unit further notifies additional information for controlling the equipment, based on another sensing result of the exhalation.

15. The exhalation sensing apparatus according to claim 13, wherein
the exhalation sensing unit senses an interval between exhalations, each of which is equivalent to the exhalation, and
the control information generation unit generates a plurality of pieces of control information, each of which is equivalent to the control information, which are different from each other, based on the interval between the exhalations.

16. The exhalation sensing apparatus according to claim 13, wherein
the exhalation sensing unit acquires a speed of change of an amount of the exhalation, and
the control information generation unit generates a plurality of pieces of control information, each of which is equivalent to the control information, which are different from each other, based on the speed of change of an amount of the exhalation.

17. The exhalation sensing apparatus according to claim 13, wherein
the exhalation sensing unit senses an interval between exhalations, each of which is equivalent to the exhalation, and acquires a speed of change of an amount of the exhalation, and
the control information generation unit generates a plurality of pieces of control information, each of which is equivalent to the control information, which are different from each other, based on a plurality of different combinations of the interval between the exhalations and the speed of change in the amount of the exhalation.

18. The exhalation sensing apparatus according to claim 13, wherein
a power portion that is the equipment, the moving object having a power portion that generates power for moving the moving object, and
the control information is information for controlling the power portion.

19. The exhalation sensing apparatus according to claim 18, wherein
the exhalation sensing unit senses alcohol included in the exhalation,
when an alcohol concentration included in the exhalation is equal to or greater than a threshold, the control information generation unit generates the control information for stopping the power portion, and
the transmitting unit transmits the control information for stopping the power portion to the power portion.

20. An exhalation sensing method performed by an exhalation sensing apparatus that is provided in an operator's cabin of a moving object and that comprises an exhalation sensing unit and a notification unit, the exhalation sensing method comprising:
sensing, by the exhalation sensing unit, an exhalation of an operator, in a state in which the operator of the moving object is not in contact therewith; and
notifying, by the notification unit, the operator of an instruction, wherein
the sensing the exhalation includes sensing, by the exhalation sensing unit, the exhalation before the notification unit notifies of the instruction, and
the notifying includes notifying, by the notification unit, the operator of an instruction based on a sensing result of the exhalation obtained by the exhalation sensing unit.

21. A non-transitory computer-readable medium having a program recorded thereon that, when performed by a computer, causes the computer to perform operations of an exhalation sensing method performed by an exhalation sensing apparatus that is provided in an operator's cabin of a moving object and that comprises an exhalation sensing unit and a notification unit, the operations comprising:
causing the exhalation sensing unit to sense an exhalation of the operator, in a state in which an operator of the moving object is not in contact therewith; and
causing the notification unit to notify the operator of an instruction, wherein
the sensing the exhalation includes sensing, by the exhalation sensing unit, the exhalation before the notification unit notifies of the instruction;
the notifying the instruction includes notifying, by the notification unit, the operator of an instruction based on a sensing result of the exhalation obtained by the exhalation sensing unit.
